(19) United States
(12) Patent Application Publication
(10) Pub. No.: US 2025/0009990 A1
(43) Pub. Date: Jan. 9, 2025

(54) DEVICES FOR INJECTING COLLAGENASE DISRUPTING AGENT(S) AND METHODS OF TREATING CONDITIONS WITH THICKENED FASCIA SUCH AS DUPUYTREN'S CONTRACTURE AND LEDDERHOSE'S DISEASE

(71) Applicant: Bradley J. Pausch, Wahpeton, ND (US)

(72) Inventor: Bradley J. Pausch, Wahpeton, ND (US)

(21) Appl. No.: 18/632,729

(22) Filed: Apr. 11, 2024

Related U.S. Application Data
(60) Provisional application No. 63/512,433, filed on Jul. 7, 2023.

(51) Int. Cl. A61M 5/42; A61M 5/19; A61M 5/32; A61M 37/00

(52) U.S. Cl. CPC A61M 5/427; A61M 5/19; A61M 5/3294; A61M 5/3297; A61M 37/0092

(57) ABSTRACT

Various embodiments of methods and devices are provided for executing a plurality of injections of a therapeutic agent into an anatomy of a patient diagnosed with Dupuytren's Contracture or Ledderhose's Disease. The plurality of injections are provided by a first therapeutic reservoir comprising a plunger configured to pressure a therapeutic agent held therein into a second, intermediate reservoir or two or more flow channels, through two or more third reservoirs, wherein each of the third reservoirs is in fluid communication with an injection needle. A preferred embodiment comprises a plurality of injection devices comprising injection needles, wherein the injection needles are arranged in a linear array.

### CROSS-REFERENCE TO RELATED APPLICATIONS

This application claims priority to provisional application No. 63/512,433, filed Jul. 7, 2023 and entitled DEVICES FOR INJECTING COLLAGENASE DISRUPTING AGENT(S) AND METHODS OF TREATING CONDITIONS WITH THICKENED FASCIA SUCH AS DUPUYTREN'S CONTRACTURE AND LEDDERHOSE'S DISEASE, the entire contents of which are incorporated herein by reference.

### STATEMENT REGARDING FEDERALLY SPONSORED RESEARCH OR DEVELOPMENT

None

### FIELD OF THE INVENTION

The present disclosure relates to devices and methods for treating Dupuytren's Contracture, Ledderhose's Disease, and/or Peyronie's disease.

### BACKGROUND

Dupuytren's disease or contracture is a thickening of the fascia (connective tissue just beneath the skin of the palm and fingers) on the palm of the hand. As the thickening progresses, lumps or nodules may form under the skin, or long thick cords of tissue may form that extend from the palm to the fingers. Often, this thickened fascia may lead to flexion deformities and involvement of one or more fingers curling to toward the palm of the hand. Such curling is known as a flexion contracture.

Dupuytren's diathesis is an aggressive form of Dupuytren's disease that affects both hands. Often, the patient with Dupuytren's diathesis also has other diseases of the fascia such as collagen nodules of the feet (Ledderhose's disease), wherein the toes of the affected regions begin to curl downward.

Symptoms of Dupuytren's disease, including Dupuytren's diathesis, initially include a painless, firm nodule under the skin of the palm. This nodule is typically in line with the ring or little fingers of the hand. As the disease progresses, the normal fascial bands may begin to thicken and may form a cord that extends from the palm to one or more of the fingers. As the cord thickens, it contracts, causing the affected fingers to begin to curl.

FIG. 1 is a photograph showing a thickened cord located along a palm of a patient's hand with Dupuytren's disease before treatment to mitigate the cord's effects.

There is no known treatment to cure or prevent Dupuytren's disease. However, surgical intervention has been used to temporarily return the affected fingers to a normal extended and non-contracted position by surgically removing the cords and/or nodules. In addition, treatments involving injection of collagenase into the thickened fascia regions have been used to weaken and/or disrupt the contracted cord, allowing the affected finger to be pulled and the cord to be stretched and/or broken to allow the finger to return to a normal extended, non-contracted position.

U.S. Pat. No. RE39,941, the contents of which are incorporated herein by reference in its entirety, discloses injection of collagenase into a Dupuytren's cord to achieve some relief. Collagenase is an enzyme with the ability to digest collagen. Currently, Xiaflex® is a currently marketed collagenase agent that is commonly injected into a Dupuytren's cord. Xiaflex® has the generic name collagenase clostridium hisolyticum and is sold as a powder that is reconstituted with a diluent. The label dosage is 0.58 mg per injection into a palpable cord. For cords affecting a metacarpophalangeal (MP) joint, the diluent amount to be used is 0.39 mL and the volume of reconstituted solution to be injected into the cord is 0.25 mL. For cords affecting a proximal interphalangeal (PIP) joint, the diluent amount to be used is 0.31 mL and the volume of reconstituted solution to be injected into the cord is 0.20 mL.

The injection of collagenase requires that the agent be injected into the site, generally along the cord, at e.g., 3 mm intervals, with separate, serial injections. Each separate injection is exceedingly painful and is one reason the patients with Dupuytren's disease do not seek treatment as often as they otherwise might. FIG. 2 is a photograph illustrating thickened cords of FIG. 1, with individual injection site locations marked at regular intervals along the affected region of the palm of the patient's hand.

If the patient with Dupuytren's disease could endure a single, parallel or simultaneous injection along a thickened cord, as opposed to multiple, serial and separate injections, the accumulated pain and anticipation of the pain during the serial injections may be lessened. In turn, the patient may seek out treatment on a more frequent and/or regular basis. Moreover, more than one individual injection requires use of a new needle for each injection.

An equivalent scenario exists in the case of Ledderhose's disease (collagen-based thickening of the plantar aspect of the foot forming nodules that, in turn, cause affected toes to curl downward). Ledderhose's disease is also currently treated, in part, with multiple, serial collagenase injections.

Peyronie's disease may also be likened to Dupuytren's disease and results in a curvature of the penis caused by plaque buildup and/or development of scar tissue in the tunica albuginea. The plaque and scar tissue do not stretch as well as normal tissue, causing a curvature of the penis. The normal tunica albuginea is composed of elastin fibers and collagen. The site of scar tissue from Peyronie's disease is composed mostly of collagen that can become calcified and, in extreme cases, harden to the thickness of bone. Peyronie's disease may be treated, in part, with therapeutic agent injections.

Various embodiments of the present invention address the issues, among others, discussed above.

### BRIEF DESCRIPTION OF THE SEVERAL VIEWS OF THE DRAWINGS

These drawings are exemplary illustrations of certain embodiments and, as such, are not intended to limit the disclosure.

FIG. 1 is a photograph of a hand of a patient with Dupuytren's disease, before undergoing a prior art series of multiple individual injections of collagenase.

FIG. 2 is a photograph of the hand of FIG. 1, with the individual injection sites marked along two thickened cords.

FIG. 3A illustrates a perspective view of one embodiment of a multi-needle injector of the present disclosure.

FIG. 3B illustrates a side cutaway view of the embodiment of FIG. 3A.

FIG. 4A illustrates a side view of one embodiment of a multi-needle injector of the present disclosure.

FIG. 4B illustrates a side view of one embodiment of a multi-needle injector of the present disclosure.

FIG. 5 illustrates an exemplary treatment method flow chart for treating Dupuytren's disease and/or Ledderhose's disease.

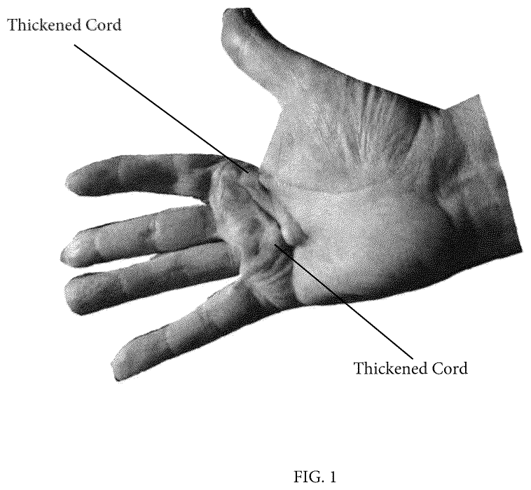

### DETAILED DESCRIPTION OF THE INVENTION

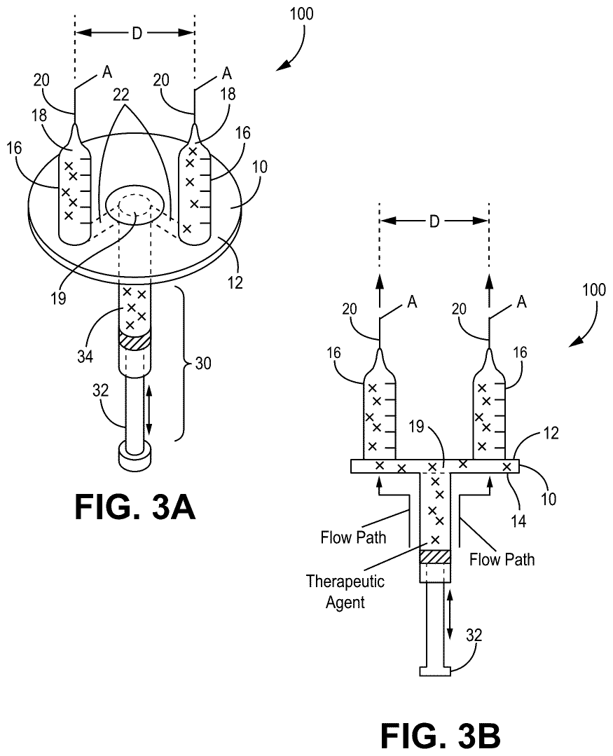

FIGS. 3A and 3B illustrate one exemplary embodiment of a multi-needle injection device 100 that may be used to execute a single, parallel set of injections in patients with, e.g., Dupuytren's disease and/or Ledderhose's disease.

As illustrated, a base 10 is provided and shown as circular, though other shapes are certainly within the scope of the present disclosure. The base 10 comprises top 12 and bottom 14 surfaces. Two injector devices 16 are shown connected to, or integrated with, the bottom surface 14 of the base 10, each injector device 16 comprising an injector reservoir 18 and an injection needle 20 at a distal end of the injector reservoir that is in fluid communication with the injector reservoir 18. Each injection needle 20 comprises a lumen (not shown but as well known in the art) that terminates at a distal aperture A. A proximal end of each injection reservoir 18 is in fluid communication with a fluid channel 22 that is defined within, and by, the base 10. A base inlet aperture 19 is defined on the top surface 12 of the base 10, wherein the base inlet aperture 19 is in fluid communication with each fluid channel 22. In some embodiments wherein the injector devices 16 are connected to the bottom surface 14 of the base 10, the injector reservoir 18, or proximal portion of the injector device 16, may be connected with the bottom surface 14 of the base 10 by connecting threads, friction fit or other known connection means such that the injector reservoir 18 is in fluid communication with the fluid channels 22.

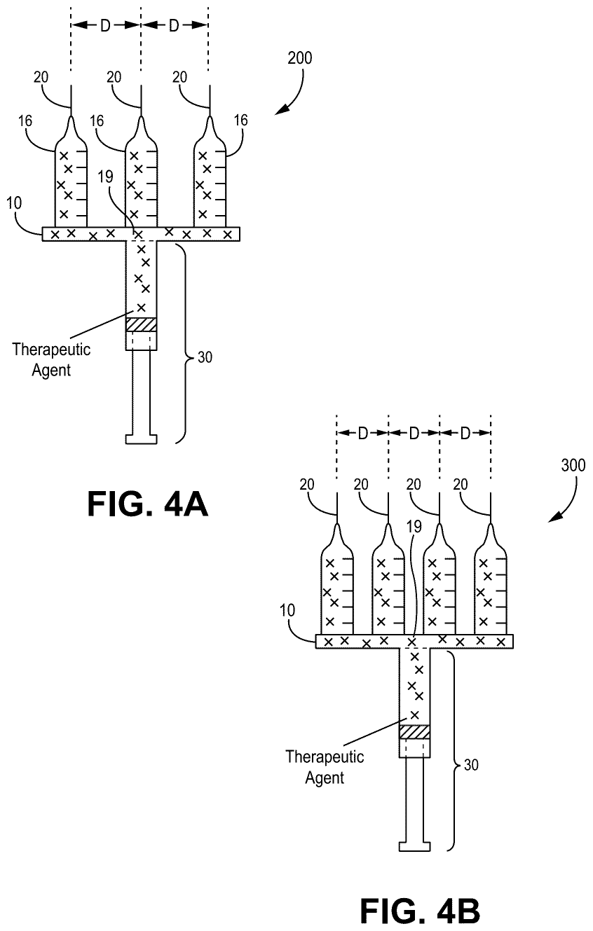

FIGS. 3A and 3B show two injector devices 16, each in fluid communication with a fluid channel 22. In other embodiments, a plurality of injector devices 16 and related fluid channels 22 may be provided. In one embodiment, e.g., three injector devices 16 spaced apart along a linear array may be provided, each injector device's reservoir 18 in fluid communication with a fluid channel, as is further discussed in relation with FIG. 4A. In other embodiments, as shown in FIGS. 3A, 4A and 4B, the injector devices 16 may comprise a plurality of injector devices 16 and may be arranged in a linear or non-linear array or pattern.

FIG. 4A illustrates an embodiment of a device 200, similar to the device of FIGS. 3A and 3B, but instead of two injector devices 16, the device 200 of FIG. 4A comprises three injector devices 16. Similarly, FIG. 4B illustrates an embodiment of a device 200, similar to the device 100 of FIGS. 3A and 3B, and the device 200 of FIG. 4A, wherein the device 300 of FIG. 4A comprises four injector devices 16. The injector devices 16 of FIGS. 4A and 4B each comprise a needle 20 spaced a distance D from adjacent needles 20.

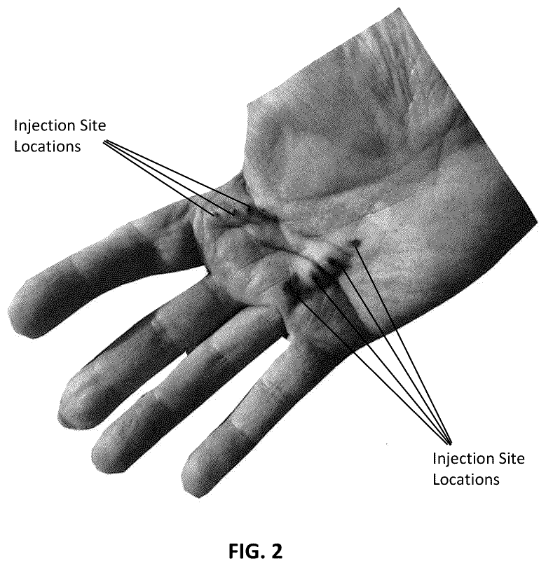

Generally, the injector devices 16 are provided in a substantially linear array to enable a single set of parallel, simultaneous injections along the substantially linear markings of FIG. 2. Adjacent injection needles 20 are spaced apart from each other along the substantially linear array at a distance D. Distance D may be about 3-4 mm, about 2-3 mm, and/or about 1-2 mm in various embodiments, and/or about 0.3-1 mm in other embodiments. A preferred distance D for Dupuytren's disease is approximately 3 mm, wherein the injection needles 20 are arranged or aligned in a substantially linear array or pattern.

In some embodiments, the spacing distance D between adjacent injection needles 20 may be substantially equivalent. In other embodiments, the spacing distance D between adjacent injection needles 20 may vary.

Injection needles 20 for Dupuytren's disease may be ½ inch and may comprise 30 gauge or 27 gauge or other gauge needle. Other size needles may be used and will be within the knowledge of the skilled artisan.

The exemplary device of FIGS. 3A-4B provide a therapeutic agent syringe 30 with a distal end that is either connected to, or integrated with, the base inlet aperture 19, and in fluid communication therewith. This allows a therapeutic agent, e.g., a collagenase, to be taken up into a reservoir 34 of the therapeutic agent syringe 30 using a plunger 32 as is well known in the art. When the targeted amount of therapeutic agent is loaded into the reservoir 34 of the therapeutic syringe 30, the distal end of the therapeutic agent syringe 30 may be connected with the base inlet aperture 19 by connecting threads, friction fit or other known connection means.

In some embodiments, the injection devices 20 may be integrated with the base 10. In these embodiments, the therapeutic agent syringe 30 may be loaded with a targeted amount of therapeutic agent and connected with the base 10 as above, and the therapeutic agent syringe plunger 32 may be pressed in a downward direction toward the top surface 12 of the base 10 to move the therapeutic agent out of the therapeutic agent syringe reservoir 34, into the base inlet aperture 19, through the respective flow channels 22 and into the injector device reservoirs 18. This process will serve to expel air from the system and allow a measured injection amount from the injector devices 20 into the patient's targeted areas.

In other embodiments, the therapeutic agent syringe 30 may be integrated with the top surface 12 of the base 10 and the injector devices 20 may be connectable with, or integrated with, the bottom surface 14 of the base 10. In these embodiments, therapeutic agent may be drawn into the injection needles 22 by placing the distal aperture A of each needle 22 into a quantity or volume of the therapeutic agent, e.g., a reconstituted volume within a container, and drawing the therapeutic agent syringe plunger 32 back away from the top surface 12, creating a suction force. In this embodiment, the reservoirs 18 of the injection devices 20 will fill with the therapeutic agent to the desired amount.

In the embodiments wherein the injector devices 20 are connectable with the base 10 as described above, the injector devices 20 and/or injection needles 22 may be removed and replaced between injections, for example when moving from one thickened cord to a second thickened cord.

Other means for filling injection devices' reservoirs may be employed including but not limited to substituting a larger reservoir within the base 10 for the individual flow channels 22, wherein the larger reservoir is in fluid communication with the injection device reservoirs 18.

Generally, the illustrated embodiments thus provide:
a first therapeutic agent reservoir (shown as an exemplary therapeutic agent reservoir 34);
a second, intermediate reservoir (shown as an exemplary base defining a reservoir, or two or more flow channels) that is in fluid communication with the first therapeutic agent reservoir; and
two or more injection reservoirs, each comprising an injection needle in fluid communication therewith) shown as exemplary injection device, the two or more injection reservoirs in fluid communication with the second, intermediate reservoir and with a distal needle.

In some embodiments, a preferred injection amount comprises a therapeutic dose and/or volume of therapeutic agent. Xiaflex®, as discussed above, is an exemplary collagenase agent that is injected to treat thickened cords of Dupuytren's disease and/or Ledderhose's disease nodules. Xiaflex® has the generic name collagenase clostridium hisolyticum and is sold as a powder that is reconstituted with a diluent. The label dosage is 0.58 mg per injection into a palpable cord. For cords affecting a metacarpophalangeal (MP) joint, the diluent amount to be used is 0.39 mL and the volume of reconstituted solution to be injected into the cord is 0.25 mL. For cords affecting a proximal interphalangeal (PIP) joint, the diluent amount to be used is 0.31 mL and the volume of reconstituted solution to be injected into the cord is 0.20 mL.

The exemplary therapeutic dosage for an exemplary collagenase such as Xiaflex® in treating patients with Dupuyten's disease may be within the range of about 0.4-0.7 mg per injection site. For cords affecting a MP joint, using the diluent volume prescribed above to reconstitute the exemplary Xiaflex® collagenase, the injection volume per injection site may be within the range of about 0.2-0.3 mL. For cords affecting a PP joint, using the diluent volume prescribe above to reconstitute the exemplary Xiaflex® collagenase, the injection volume per injection site may be within the range of about 0.15 to 0.25 mL.

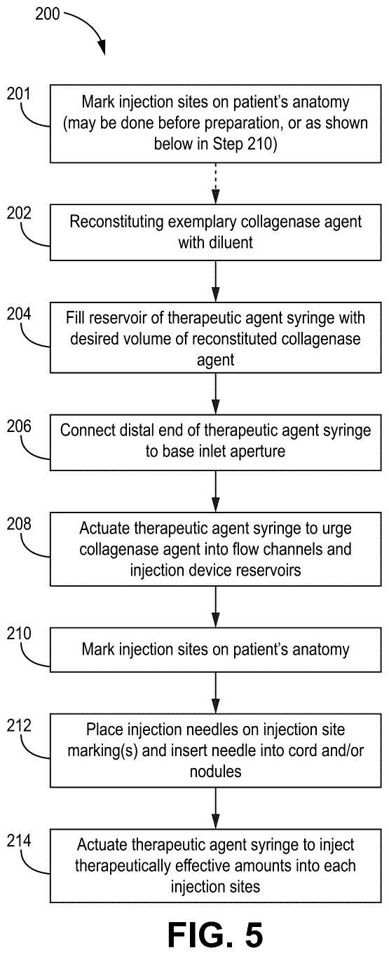

An exemplary method for disrupting cords found in Dupuytren's disease patients, and/or nodules found in Ledderhose's disease patients, is illustrated in FIG. 5.

The exemplary treatment method 200 may be initiated by optionally marking injection targets or sites on the patient's anatomy at step 201. Alternatively, the marking of injection targets may be done later in the method, for example, at step 210, though the marking may be done at any point prior to the injection steps of 212 and 214.

Next, at step 202, a desired dose and volume of an exemplary collagenase agent, or other therapeutic agent or composition, may be prepared or reconstituted. Next, at step 204, a reservoir 34 of a therapeutic agent syringe 30 may be filled with a desired volume of the reconstituted exemplary collagenase agent. At step 206, the distal end of the therapeutic agent syringe 30 may be connected with the base inlet aperture 19 of the base 10 as shown in FIGS. 3A and 3B. Next, in step 208, the plunger 32 of the therapeutic agent syringe 30 may be actuated toward the top surface 12 of the base 10 to urge the therapeutic agent out of the reservoir 34 of the therapeutic agent syringe 30, through two or more flow channels 22 defined by, or within, the base 10 and into two or more injector device reservoirs 18 connected to or integrated with, the bottom surface 14 of base 10 as shown in FIGS. 3A and 3B and as described above. As noted, the distal end of the therapeutic agent syringe 30 may be connected with the base inlet aperture 19 by connecting threads, friction fit or other known connection means.

The patient's hands (or feet) may be marked at any point in the process at regular intervals, or with a line, along a substantially linear path, for example at steps 201, 210, or other. In step 212, the injection needles 20 are placed on the markings, or along the line, and used to pierce the skin and enter the cord at the marked desired locations. Step 214 comprises further actuation of the therapeutic agent syringe plunger 32 to inject a therapeutic amount of the exemplary collagenase into the patient's cord and/or nodules via the injection needles 20.

The above exemplary procedure may be repeated if additional cords and/or length along a cord requires injections.

Thus, the exemplary procedure requires a single set of a plurality of injections, all completed simultaneously.

In some embodiments, a calcium channel blocker may be added to the procedure, either as a separate injection treatment, or combined with the exemplary collagenase to create a pharmaceutic composition that is injected as described herein. For example, an L-type phenyl-alkylamine class calcium channel blocker such as Verapamil may be provided and injected to, among other things, slow the progression of reformation of the cords and/or nodules. A dose range of such a calcium channel blocker may be within the range of about 0.4 mg to about 3.5 mg. In other embodiments, the dose range of the calcium channel blocker may be within the range of about 0.3 to about 4.5 mg.

Further, low intensity shockwave treatment may be applied to the affected areas of the hands and/or feet to soften the cords and/or nodules. This low intensity shockwave treatment maybe done at a point that is before the exemplary collagenase, or related pharmaceutical composition is injected according to the present disclosure and/or after the injection is completed. One or more of the shockwave treatments may be provided and the shockwaves may have a maximum energy of about 0.09 mJmm². US Publication US-2015-0073312, filed by Ein-gal, discloses one method of low intensity shockwave treatment and is incorporated by reference herein for the purpose of disclosing low intensity shockwave treatment. In an exemplary treatment, about 300 pulses may be applied per minute to the affected area(s) over the course of between about 10 and 20 minutes.

The multi-needle injection devices described herein, and the method of use of the multi-needle injection devices described herein, may be used to execute a single set of parallel, simultaneous injections. These injections may be delivered by a plurality of injection needles 20 that are spaced apart from each other. In a preferred embodiment, the injection needles 20 may be spaced apart along a linear path. In other embodiments, a non-linear path may be provided by the injection needles.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

Having described the invention, we claim:

1. A method for treating Dupuytren's disease and/or Ledderhose's disease and/or Peyronie's disease, comprising:
marking a plurality of desired injection locations on the patient's anatomy;
providing a multi-needle injector comprising a linear, or non-linear, array of two or more injection devices, each injection device comprising an injection reservoir and an injection needle;
providing a collagenase agent;
ensuring that a therapeutic dose of the collagenase agent is proved within the two or more injection reservoirs;
placing the array of injection needles at or along the marked desired injection locations; and
actuating the multi-needle injector to simultaneously inject a therapeutically effective amount of the collagenase agent into the patient's anatomy at a plurality of the marked plurality of desired injection locations.

2. The method of claim 1, wherein adjacent ones of the two or more injection needles are spaced apart by a distance of 3-4 mm.

3. The method of claim 1, wherein adjacent ones of the injection needles are spaced apart by a distance of 2-3 mm.

4. The method of claim 1, wherein adjacent ones of the two or more injection needles are spaced apart by a distance of 1-2 mm.

5. The method of claim 1, wherein adjacent ones of the two or more injection needles are spaced apart by a distance of 0.3-1 mm.

6. The method of claim 1, further comprising injecting a therapeutically effective amount of a calcium channel blocker before, during and/or after the collagenase injection is completed.

7. The method of claim 1, further comprising injecting a pharmaceutical composition comprising the collagenase agent and the calcium channel blocker into the marked desired target locations with the multi-needle injector.

8. The method of claim 1, further comprising applying one or more low intensity shockwave treatments to the before and/or after the collagenase agent injecting is completed.

9. The method of claim 7, further comprising applying one or more low intensity shockwave treatments to the before and/or after the pharmaceutical composition injecting is completed.

10. A multi-injector device for use in treating Dupuytren's Contracture or Ledderhose's Disease with simultaneous and spaced-apart injections of a therapeutic agent, comprising:
a first therapeutic agent reservoir comprising a plunger operatively associated with the first therapeutic agent reservoir;
a second, intermediate reservoir in fluid communication with the first therapeutic agent reservoir; and
two or more injection reservoirs, each one of the two or more injection reservoirs comprising an injection needle comprising a lumen and distal aperture in fluid communication and operative association therewith,
wherein adjacent ones of the two or more injection reservoirs and associated injection needles are spaced apart a distance and wherein the two or more injection reservoirs and associated injection needles are arranged in a linear pattern,
wherein the plunger is configured to pressure a therapeutic agent from the first therapeutic agent reservoir into the second, intermediate reservoir and into each one of the two or more injection reservoirs,
wherein the plunger is further configured to pressure the therapeutic agent from each one of the two or more injection reservoirs and through the associated injection needle's lumen and distal aperture.

11. The multi-injector device of claim 10, wherein adjacent ones of the two or more injection needles are spaced apart by a distance of 3-4 mm.

12. The method of claim 10, wherein adjacent ones of the injection needles are spaced apart by a distance of 2-3 mm.

13. The method of claim 10, wherein adjacent ones of the two or more injection needles are spaced apart by a distance of 1-2 mm.

14. The method of claim 10, wherein adjacent ones of the two or more injection needles are spaced apart by a distance of 0.3-1 mm.

15. A multi-injector device for use in treating Dupuytren's Contracture or Ledderhose's Disease with simultaneous spaced-apart injections of a therapeutic agent, comprising:
a therapeutic agent syringe comprising a therapeutic agent reservoir configured to hold at least a therapeutic dose of the therapeutic agent, the therapeutic agent reservoir comprising a distal end and a plunger in fluid communication and operative association with the therapeutic agent reservoir and configured to pressure the therapeutic agent out of the distal end of the therapeutic agent reservoir;
a base having a top surface and a bottom surface, the bottom surface in fluid communication with the distal end of the therapeutic agent reservoir;
two or more flow channels defined by the base and in fluid communication with the distal end of the therapeutic agent reservoir;
two or more injector devices,
wherein each one of the injector devices comprises:
an injector reservoir in fluid communication with one of the flow channels, and
an injection needle in fluid communication with the injector reservoir, the needle comprising a lumen therethrough and a distal aperture,
wherein the two or more injection needles are arranged in a pattern.

16. The multi-injector device of claim 15, wherein the two or more injection needles are arranged in a linear array, wherein adjacent ones of the two of more injection needles are spaced apart from each other by a distance.

17. The multi-injector device of claim 16, wherein adjacent ones of the two or more injection needles are spaced apart by a distance of 3-4 mm.

18. The method of claim 16, wherein adjacent ones of the injection needles are spaced apart by a distance of 2-3 mm.

19. The method of claim 16, wherein adjacent ones of the two or more injection needles are spaced apart by a distance of 1-2 mm.

20. The method of claim 16, wherein adjacent ones of the two or more injection needles are spaced apart by a distance of 0.3-1 mm.
